(19) United States
(12) Patent Application Publication
(10) Pub. No.: US 2003/0021841 A1
(43) Pub. Date: Jan. 30, 2003

(54) Pharmaceutical composition

(76) Inventors: Amol Singh Matharu, Cranbury, NJ (US); Mahendra R. Patel, East Brunswick, NJ (US)

Correspondence Address: THOMAS HOXIE, NOVARTIS CORPORATION, PATENT AND TRADEMARK DEPT, 564 MORRIS AVENUE, SUMMIT, NJ, 079011027

(21) Appl. No.: 10/183,881

(22) Filed: Jun. 27, 2002

Related U.S. Application Data
(60) Provisional application No. 60/302,613, filed on Jul. 2, 2001.

(51) Int. Cl. A61K 9/20; B27N 3/00

(52) U.S. Cl. 424/465; 264/109

(57) ABSTRACT

The present invention relates to a process for preparing tablet dosage forms of poorly-compressible pharmaceutical agents and to tablet dosage forms prepared according to the inventive process. The inventive process is especially useful for preparing tablets of the poorly-compressible drug metformin HCl.

### SUMMARY

The present invention relates to a process for preparing a pharmaceutical tablet formulation of a poorly-compressible pharmaceutical agent, for example, the drug metformin HCl formulated as a monolithic or single phase homogenous system.

### BACKGROUND

Some pharmaceutical agents are difficult to formulate into a tablet dosage form due to agent's poor compressibility. Conventional tablet formulations of such poorly-compressible pharmaceutical agents lack adequate hardness and are often friable. Thus, special formulation techniques are required to formulate poorly-compressible pharmaceutical agents into a commercially viable tablet dosage form.

One way to overcome the poor compressibility of pharmaceutical agents is to utilize wet granulation techniques to prepare the tablet formulation. This involves additional unit operations of wet milling, drying and milling of dried granulation. However, tablets prepared by wet methods often show incremental hardness as a function of time and storage temperature. Therefore, tablets prepared by wet methods are more likely to show variable product performance.

The object of the present invention is to prepare pharmaceutical tablets of poorly-compressible drugs that have adequate and stable hardness and good reproducibility, by a process that avoids wet granulation during processing. This object is achieved by formulating the poorly-compressible pharmaceutical agent according to a process whereby the active ingredient, an erodible hydrophilic component, a hydrophobic component and optionally other excipients, are blended, and the resulting blend is sized and/or lubricated, if necessary, and compressed into tablets as a monolithic or single-phase homogenous system.

The present invention is applicable to non-compressible drugs and drugs susceptible to hydrolysis and degradation due to water or a solvent.

### DETAILED DESCRIPTION

The present invention relates to a process for preparing a pharmaceutical tablet formulation of a poorly-compressible pharmaceutical agent, which comprises:

(a) preparing a blend by combining the poorly-compressible pharmaceutical agent, a hydrophilic erodible component and a hydrophobic component as a monolithic or single phase homogenous system; and

(b) compressing the blend into a tablet.

The blend may optionally comprise other pharmaceutically acceptable excipients and the blend may optionally be lubricated prior to being compressed into tablets. The present invention is applicable to non-compressible drugs and drugs susceptible to hydrolysis and degradation due to water or a solvent.

A poorly compressible substance is one that does not bond to form a tablet upon application of compression force. Therefore, such substances may require additional processing and special formulating before it can be compressed into a tablet. With such substances, the additional processing necessary is usually a wet granulation step; direct compression would not be effective. These substances may also be formulated with binders or other materials having high binding capacity (or act as an aid to compressibility) such that the non-bonding properties of the non-compressible material is overcome. Other techniques to assist compression include having residual moisture in the blend prior to compression or having the non-compressible material in very low amounts in the tablet formulation. High-dose drugs do not lend themselves to direct compression because of poor flowability and poor compressibility.

The hydrophilic erodible component of the present invention is a pharmaceutically acceptable excipient which is a water-loving soluble/gellable agent. These components possess properties, such as the ability to imbibe external fluid and dissolve/erode over a period of time. Typical hydrophilic erodible components include hydroxypropylmethyl cellulose; soluble fillers, such as lactose; tablet disintegrants, such as croscarmellose sodium; binders, such as polyvinylpyrrolidone; gums, such as guar and xanthan gums. Examples of water soluble and/or swellable hydrophilic polymers include solid polyethylene glycol with molecular weights greater than 400(MW>400), celluloses (hydroxymethyl cellulose, hydroxypropyl cellulose, hydroxyethyl cellulose, carboxymethyl cellulose, carboxyethyl cellulose, sodium carboxymethyl cellulose, sodium alginate, methyl cellulose, hydroxypropyl methyl cellulose), carboxypolymethylene, gums (acacia gum, guar gum, tragencanth gum and xanthan gum), polyethylene oxide and the like. High molecular weight cellulose derivatives are preferred as the hydrophilic erodible component.

The hydrophobic component is a pharmaceutically acceptable excipient which is water insoluble and does not dissolve in water over a period of time. Typical hydrophobic components include ethyl cellulose, methacrylic acid polymers and copolymers, such as EUDRAGIT NE 30 D from Rohm and Haas, fatty acids and esters thereof, such as stearic acid, behenic acid, glyceryl monostearate, glyceryl palmitostearate, glyceryl behenate and other waxes, such as carbuna wax. Also included are high molecular weight fatty alcohols, such as cetyl alcohol and the like. Cetyl alcohol and stearyl alcohol are preferred as the hydrophobic component.

The poorly-compressible pharmaceutical agent typically represents from about 10% to about 90% by weight of the formulation. Preferably, the poorly-compressible pharmaceutical agent is present in the formulation in amount of from about 30% to about 70% by weight.

The hydrophilic erodible component typically represents from about 10% to about 90% by weight of the formulation. Preferably, the hydrophilic erodible component is present in the formulation in amount of from about 30% to about 70% by weight.

The hydrophobic component typically represents from about 1% to about 30% by weight of the formulation. Preferably, the hydrophobic component is present in the formulation in amount of from about 15% to about 25% by weight.

Typically, the ratio of hydrophilic erodible component to hydrophobic component is in the range from about 9:1 to 1:1. Preferably the ratio is in the range from about 2:1 to 3:1.

Preferred formulations comprise from about 40% to about 60% by weight of the poorly-compressible pharmaceutical agent and comprise the hydrophilic erodible component and hydrophobic component in a ratio of from about 2:1 to 3:1.

The poorly-compressible pharmaceutical agent, hydrophilic erodible component and hydrophobic component are blended by standard techniques. Typically, the components are added to a standard blending apparatus and blended. Hydrophobic components which are solid at room temperature, such as waxes, are often liquefied before and/or during the blending operation.

In another embodiment, the poorly-compressible pharmaceutical agent and hydrophilic erodible component are pre-mixed by standard techniques and then combined with the hydrophobic component. The pre-mixed components are combined with the hydrophobic component by a variety of techniques, such as adding the hydrophobic component to the blending apparatus containing the pre-mixed components. The fluidized bed technique may also be used and is especially appropriate when the hydrophobic component is ethyl cellulose or a polymethacrylic acid polymer or co-polymer.

The blend produced by combining the poorly-compressible pharmaceutical agent, hydrophilic erodible component and hydrophobic component is typically a monolithic or single phase homogenous free flowing powder. As is typical when formulating tablets, the free flowing powder blend is often milled or sieved in order to control the particle size of the blend and to remove large agglomerates.

If needed, the blend my optionally be lubricated prior to compression into tablets. Typical lubricants include magnesium stearate and stearic acid. However, the presence of the hydrophobic component often renders additional lubrication unnecessary. Additional lubricants will generally represent 0% to about 6% by weight of the tablet formulation.

In addition to the poorly-compressible pharmaceutical agent, hydrophilic erodible component, hydrophobic component and optional lubricant, the tablet formulations of the present invention may contain additional pharmaceutical excipients, such as flavoring agents, binders and/or fillers.

Since an objective of the present invention is to form a compressible formulation by a process other than wet granulation, the granulation process will be conducted without solvent or water. It is preferred for the process to be carried out under substantially anhydrous conditions.

The process of the present invention is useful for preparing both immediate-release and sustained-release tablet dosage forms of poorly-compressible pharmaceutical agents. The release rate of the pharmaceutical agent is controlled by the hydrophilic erodible agent and hydrophobic agent. Thus, an immediate-release formulation will typically contain the hydrophilic erodible component and hydrophobic components in a ratio of from about 1:9 to 2:8. Increasing the amount of hydrophilic erodible component will extend the release rate of the pharmaceutical agent. Thus, sustained-release tablet dosage forms typically contain the hydrophilic erodible component and hydrophobic components in a ratio of from about 3:1 to 2:1.

The dissolution profile obtained in phosphate buffer (pH 6.8), USP Apparatus II, for tablets prepared according to the present invention are:

| Time (hrs) | % Dissolved |
|---|---|
| 1 | 20-40 |
| 4 | 50-70 |
| 7 | 75-90 |

Examples of poorly-compressible pharmaceutical agents that are formulated into tablets in accordance with the inventive process include metformin HCl, naproxen or naproxen sodium. High-dose drugs do not lend themselves to direct compression because of poor flowability and poor compressibility. Representative active medicaments include antacids, anti-inflammatory substances, coronary dilators, cerebral dilators, peripheral vasodilators, anti-infectives, psychotropics, antimanics, stimulants, antihistamines, laxatives, decongestants, vitamins, gastrointestinal sedatives, antidiarrheal preparations, anti-anginal drugs, vasodilators, antiarrythmics, anti-hypertensive drugs, vasoconstrictors and migraine treatments, anticoagulants and antithrombotic drugs, analgesics, anti-pyretics, hypnotics, sedatives, anti-emetics, anti-nauseants, anti-convulsants, neuromuscular drugs, hyper-and hypoglycemic agents, thyroid and anti-thyroid preparations, diuretics, anti-spasmodics, uterine relaxants, mineral and nutritional additives, anti-obesity drugs, anabolic drugs, erythropoietic drugs, anti-asthmatics, expectorants, cough suppressants, mucolytics, anti-uricemic drugs, and drugs or substances acting locally in the mouth.

Typical active medicaments include gastrointestinal sedatives, such as metoclopramide and propantheline bromide; antacids, such as aluminum trisilicate, aluminum hydroxide and cimetidine; anti-inflammatory drugs, such as phenylbutazone, indomethacin, naproxen, ibuprofen, flurbiprofen, diclofenac, dexamethasone, prednisone and prednisolone; coronary vasodilator drugs, such as glyceryl trinitrate, isosorbide dinitrate and pentaerythritol tetranitrate; peripheral and cerebral vasodilators, such as soloctidilum, vincamine, naftidrofuryl oxalate, co-dergocrine mesylate, cyclandelate, papaverine and nicotinic acid; anti-infective substances, such as erythromycin stearate, cephalexin, nalidixic acid, tetracycline hydrochloride, ampicillin, flucolaxacillin sodium, hexamine mandelate and hexamine hippurate; neuroleptic drugs, such as fluazepam, diazepam, temazepam, amitryptyline, doxepin, lithium carbonate, lithium sulfate, chlorpromazine, thioridazine, trifluperazine, fluphenazine, piperothiazine, haloperidol, maprotiline hydrochloride, imipramine and desmethylimipramine; central nervous stimulants, such as methylphenidate, ephedrine, epinephrine, isoproterenol, amphetamine sulfate and amphetamine hydrochloride; anti-histamic drugs such as diphenhydramine, diphenylpyraline, chlorpheniramine and brompheniramine; anti-diarrheal drugs, such as bisacodyl and magnesium hydroxide; the laxative drugs, such as dioctyl sodium sulfosuccinate; nutritional supplements, such as ascorbic acid, alpha tocopherol, thiamine and pyridoxine; anti-spasmotic drugs, such as dicyclomine and diphenoxylate; drugs effecting the rhythm of the heart, such as verapamil, nifedepine, diltiazem, procainamide, disopyramide, bretylium tosylate, quinidine sulfate and quinidine gluconate; drugs used in the treatment of hypertension, such as propranolol hydrochloride, guanethidine monosulphate, methyldopa, oxprenolol hydrochloride, captopril and hydralazine; drugs used in the treatment of migraine, such as ergotamine; drugs effecting coagulability of blood, such as epsilon aminocaproic acid and protamine sulfate; analgesic drugs, such as acetylsalicylic acid, acetaminophen, codeine phosphate, codeine sulfate, oxycodone, dihydrocodeine tartrate, oxycodeinone, morphine, heroin, nalbuphine, butorphanol tartrate, pentazocine hydrochloride, cyclazacine, pethidine, buprenorphine, scopolamine and mefenamic acid; anti-epileptic drugs, such as phenytoin sodium and sodium valproate; neuromuscular drugs, such as dantrolene sodium; substances used in the treatment of diabetes, such as tolbutamide, diabenase glucagon and insulin; drugs used in the treatment of thyroid gland disfunction, such as triiodothyronine, thyroxine and propylthiouracil; diuretic drugs, such as furosemide, chlorthalidone, hydrochlorthiazide, spironolactone and triampterene; uterine relaxant drugs, such as ritodrine; appetite suppressants, such as fenfluramine hydrochloride, phentermine and diethylproprion hydrochloride; anti-asthmatic drugs, such as aminophylline, theophylline, salbutamol, orciprenaline sulphate and terbutaline sulphate, expectorant drugs, such as guaiphenesin; cough suppressants, such as dextromethorphan and noscapine; mucolytic drugs, such as carbocisteine; anti-septics, such as cetylpyridinium chloride, tyrothricin and chlorhexidine; decongestant drugs, such as phenylpropanolamine and pseudoephedrine; hypnotic drugs, such as dichloralphenazone and nitrazepam; anti-nauseant drugs, such as promethazine theoclate; haemopoetic drugs, such as ferrous sulphate, folic acid and calcium gluconate, uricosuric drugs, such as sulphinpyrazone, allopurinol and probenecid and the like.

The invention is applicable to sublingual lozenges, buccal tablets, oral lozenges, suppositories and compressed tablets, the latter being intended to be swallowed in unit dosage form and which upon ingestion according to a prescribed regimen give slow and regular release of active medicaments of a fixed percentage in the intestinal tract. It is further understood that the invention is not restricted to the above listed medications.

The process of the present invention is especially useful for formulating metformin HCl into tablets. Thus, the present invention especially relates to a process wherein the poorly-compressible pharmaceutical agent is selected from the group consisting of mefformin HCl.

In a preferred embodiment, the poorly-compressible pharmaceutical agent is metformin HCl, the hydrophilic erodible component is hydroxypropyl methylcellulose and the hydrophobic component is stearyl alcohol, wherein the hydrophilic agent and hydrophobic agent are in a ratio of from about 3:1 to about 2:1.

Preferably, the tablet comprises from about 40-60% by weight of metformin HCl.

Most preferably, the tablet comprises from about 45-50% by weight of metformin HCl, a hydrophilic erodible component which is hydroxypropyl methylcellulose and a hydrophobic component which is stearyl alcohol and a weight ratio of hydrophilic erodible component to hydrophobic component in the range from 3:1 to about 2:1.

In one embodiment, the tablet may comprise 250 mg, 500 mg, 850 mg or 1 g of metformin HCl. Most preferably, the tablet comprises about 500 mg of metformin HCl.

The present invention further relates to pharmaceutical tablet dosage form which comprises a poorly-compressible pharmaceutical agent, a hydrophilic erodible component and a hydrophobic component. All of the preferences discussed above for the process also apply to the tablet dosage form, if applicable to a tablet dosage form.

The present invention further relates to a pharmaceutical tablet dosage form of metformin HCl with the preferences discussed above applying to the dosage form.

### EXAMPLE 1

| Item no. | Ingredient | % | mg/unit | Amount per batch |
|---|---|---|---|---|
| 1 | Metformin HCl | 48.54 | 500 | 55 kg |
| 2 | Hydroxypropyl | 31.06 | 320 | 39.05 kg |
| | methylcellulose | | | |
| 3 | Stearyl alcohol | 19.41 | 200 | 18.15 kg |
| 4 | Magnesium stearate | 0.97 | 10 | 1.1 kg |
| | Total | 100 | 1030 | 113.3 kg |

Metformin HCl is first de-lumped using Fitz-mill equipped with 0.050″ screen at Medium speed. De-lumped metformin HCl and hydroxypropyl methyl cellulose (available as Methocel K100 Premium CR, Dow Chemical Company, MI) are mixed in a 340 Qt. AMF Planetary Mixer and mixed for 10 minutes to form a pre-mix blend. The pre-mix blend is transferred to drums. To a pre-heated jacketed bowl of 340 Qt. AMF Planetary Mixer, stearyl alcohol is added and allowed to melt to form a clear liquid at the jacket temperature of not less than 65° C. To the melted wax, pre-mix is added and mixed until a uniform granulation is obtained while heating at the jacket temperature of not less than 65° C. The granulation is transferred to trays lined with Kraft paper and cooled down to a temperature of 25° C.-30° C. The cooled granulation is sized using a low energy screening/milling device such as a Glatt Quick sieve equipped with 1.5 mM screen. The lubrication is performed using magnesium stearate in a 30 cu. ft. Gemco Blender. The final-mix obtained is compressed into tablets using Manesty Unipress Diamond using modified oval tools. The hardness of the tablets obtained was 10-18 SCU. The dissolution profile of the tablets matched that of Glucophage® XR500 mg (Bristol-Myers Squibb, NJ).

The dissolution profile (average, n=6) obtained in phosphate buffer (pH 6.8), USP Apparatus II, is:

| Time (hours) | % dissolved |
|---|---|
| 1 | 31.7 |
| 4 | 64.2 |
| 7 | 80.1 |

### EXAMPLE 2

| Item no. | Ingredient | % | mg/unit | Amount per batch |
|---|---|---|---|---|
| 1 | Metformin HCl with | 48.8 | 502.5 | 40.0 kg |
| | 0.5% magnesium | | | |
| | stearate | | | |
| 2 | Hydroxypropyl | 28.8 | 297 | 23.76 kg |
| | methylcellulose | | | |
| 3 | Stearyl alcohol | 21.4 | 220 | 17.6 kg |
| 4 | Colloidal silicon dioxide | 0.3 | 3 | 0.24 kg |
| 5 | Magnesium stearate | 0.7 | 7.5 | 0.6 kg |
| | TOTAL | 100 | 1030 | 82.4 kg |

Metformin HCl with 0.5% magnesium stearate and hydroxypropyl methyl cellulose (avaliable as Methocel K100 M Premium CR, Dow Chemical Company, MI) are mixed in a PMA 300 Fielder High Shear to form a pre-mix blend. The pre-mix blend is transferred to drums. To a pre-heated jacketed bowl of 340 Qt. AMF Planetary Mixer, stearyl alcohol is added and allowed to melt to form a clear liquid at the jacket temperature of not less than 65° C. To the melted wax, pre-mix is added and mixed until a uniform granulation is obtained while heating at the jacket temperature of not less than 65° C. The granulation is transferred to trays lined with Kraft paper and cooled down to a temperature of 25° C.-30° C. The cooled granulation is sized using a low energy screening/milling device such as a Quadro Co-Mill equipped with 93 screen. The Pre-lubrication and Lubrication is performed using colloidal silicon dioxide and magnesium stearate, respectively, in a Patterson-Kelley Blender. The final-mix obtained is compressed into tablets using Manesty Unipress Diamond using modified oval tools. The hardness of the tablets obtained was 10-18 SCU. The dissolution profile of the tablets matched that of Glucophagee XR 500 mg (Bristol-Myers Squibb, NJ).

The dissolution profile (average, n=6) obtained in phosphate buffer (pH 6.8), USP Apparatus II, is:

| Time (hours) | % dissolved |
|---|---|
| 1 | 31.5 |
| 4 | 63.5 |
| 7 | 80.9 |

### EXAMPLE 3 (Immediate-Release Formulation)

| Item no. | Ingredient | % | mg/unit |
|---|---|---|---|
| 1 | Metformin HCl | 71.4 | 500 |
| 2 | Hydroxypropyl methylcellulose | 10.6 | 74 |
| 3 | Stearyl alcohol | 17.0 | 119 |
| 4 | Colloidal silicon dioxide | 0.2 | 1.4 |
| 5 | Magnesium stearate | 0.8 | 5.6 |
| | TOTAL | 100 | 700 |

Metformin HCl and hydroxypropyl methyl cellulose (available as Pharmacoat 606, Shin-Etsu Chemical Co. Ltd., Japan) are mixed in a 500 mL glass beaker with the help of a stainless steel spatula. Stearyl alcohol is melted in a glass beaker. To the melted wax, pre-mix is added and mixed until a uniform granulation is obtained while heating at temperature of not less than 65° C. The granulation is transferred to Kraft paper and cooled down to a temperture of 25° C.-30° C. The cooled granulation is sized using screen #20. The Pre-lubrication and Lubrication is performed using colloidal silicon dioxide and magnesium stearate, respectively, in a glass beaker using a stainless steel spatula. The final-mix obtained compressed into tablets using Carver hydraulic press. The hardness of the tablets obtained was 8 SCU.

The dissolution profile (average, n=3) obtained in phosphate buffer (pH 6.8), USP Apparatus II, is:

| Time (minutes) | % dissolved |
|---|---|
| 15 | 43.9 |
| 30 | 76.1 |
| 45 | 97.1 |
| 60 | 100.2 |

### EXAMPLE 4 (Immediate-Release Formulation)

| Item no. | Ingredient | % | mg/unit |
|---|---|---|---|
| 1 | Metformin HCl | 50.0 | 500 |
| 2 | Microcrystalline cellulose | 32.0 | 320 |
| 3 | Stearyl alcohol | 17.0 | 170 |
| 4 | Colloidal silicon dioxide | 0.2 | 2 |
| 5 | Magnesium stearate | 0.8 | 8 |
| | TOTAL | 100 | 1000 |

Metformin HCl and microcrystalline cellulose are mixed in a 500 mL glass beaker with the help of a stainless steel spatula. Stearyl alcohol is melted in a glass beaker. To the melted wax, pre-mix is added and mixed until a uniform granulation is obtained while heating at temperature of not less than 65° C. The granulation is transferred to Kraft paper and cooled down to a temperature of 25° C.-30° C. The cooled granulation is sized using screen No. 20 The pre-lubrication and lubrication is performed using colloidal silicon dioxide and magnesium stearate, respectively, in a glass beaker using a stainless steel spatula. The final-mix obtained is compressed into tablets using Carver hydraulic press. The hardness of the tablets obtained was 8 SCU.

The dissolution profile (average, n=3) obtained in phosphate buffer (pH 6.8), USP Apparatus II, is:

| Time (minutes) | % dissolved |
|---|---|
| 5 | 75.6 |
| 15 | 100.4 |

We claim:

1. A process for preparing a pharmaceutical tablet formulation of a poorly-compressible pharmaceutical agent, which comprises the steps of
(a) preparing a blend by combining the poorly-compressible pharmaceutical agent, a hydrophilic erodible component and a hydrophobic component; and
(b) compressing the blend into a tablet.

2. A process accordingly to claim 1 further comprising mixing an optional lubricant with the blend prior to compressing the blend into a tablet.

3. A process accordingly to claim 1 further comprising mixing optional pharmaceutically acceptable excipients with the blend prior to compressing the blend into a tablet.

4. A process according to claim 1, wherein the process is carried out under substantially anhydrous conditions.

5. A process according to claim 1, wherein the poorly-compressible pharmaceutical agent is selected from the group consisting of metformin HCl, metoclopramide, propantheline bromide, aluminum trisilicate, aluminum hydroxide, cimetidine, phenylbutazone, indomethacin, naproxen, ibuprofen, flurbiprofen, diclofenac, dexamethasone, prednisone and prednisolone, glyceryl trinitrate, isosorbide dinitrate, pentaerythritol tetranitrate, soloctidilum, vincamine, naftidrofuryl oxalate, co-dergocrine mesylate, cyclandelate, papaverine, nicotinic acid, clarithromycin, azithromycin, erythromycin stearate, cephalexin, nalidixic acid, tetracycline hydrochloride, ampicillin, flucolaxacillin sodium, hexamine mandelate, hexamine hippurate, fluazepam, diazepam, temazepam, amitryptyline, doxepin, lithium carbonate, lithium sulfate, chlorpromazine, thioridazine, trifluperazine, fluphenazine, piperothiazine, haloperidol, maprotiline hydrochloride, imipramine and desmethylimipramine, methylphenidate, ephedrine, epinephrine, isoproterenol, amphetamine sulfate and amphetamine hydrochloride, diphenhydramine, diphenylpyraline, chlorpheniramine and brompheniramine, bisacodyl, magnesium hydroxide, dioctyl sodium sulfosuccinate, ascorbic acid, alpha tocopherol, thiamine, pyridoxine, dicyclomine, diphenoxylate, verapamil, nifedepine, diltiazem, procainamide, disopyramide, bretylium tosylate, quinidine sulfate, quinidine gluconate, propranolol hydrochloride, guanethidine monosulphate, methyldopa, oxprenolol hydrochloride, captopril and hydralazine, ergotamine, epsilon aminocaproic acid, warfarinm sodium, ticlopidine, protamine sulfate, acetylsalicylic acid, acetaminophen, codeine phosphate, codeine sulfate, oxycodone, dihydrocodeine tartrate, oxycodeinone, morphine, heroin, nalbuphine, butorphanol tartrate, pentazocine hydrochloride, cyclazacine, pethidine, buprenorphine, scopolamine and mefenamic acid, phenytoin sodium and sodium valproate, dantrolene sodium, tolbutamide, diabenase glucagons, glipizide, glyburide, insulin, triiodothyronine, thyroxine and propylthiouracil, furosemide, chlorthalidone, hydrochlorthiazide, spironolactone, triampterene, ritodrine, fenfluramine hydrochloride, phentermine and diethylproprion hydrochloride, aminophylline, theophylline, salbutamol, orciprenaline sulphate, terbutaline sulphate, guaiphenesin, dextromethorphan, noscapine, carbocisteine, cetylpyridinium chloride, tyrothricin and chlorhexidine, phenylpropanolamine and pseudoephedrine; hypnotic drugs, such as dichloralphenazone, nitrazepam, promethazine theoclate, ferrous sulphate, folic acid and calcium gluconate, sulphinpyrazone, allopurinol and probenecid and the like.

6. A process according to claim 1, wherein the poorly-compressible pharmaceutical agent is selected from the group consisting of metformin HCl.

7. A process according to claim 1, wherein the hydrophilic erodible component is selected from the group consisting of hydroxypropyl methylcellulose, lactose, croscarmellose sodium, polyvinylpyrrolidone, guar and xanthan gums, polyethylene glycol (MW>400), celluloses, hydroxymethyl cellulose, hydroxypropyl cellulose, hydroxyethyl cellulose, carboxymethyl cellulose, carboxyethyl cellulose, sodium carboxymethyl cellulose, sodium alginate, methyl cellulose, carboxypolymethylene, acacia gum, tragencanth gum and polyethylene oxide.

8. A process according to claim 7, wherein the hydrophilic erodible component is hydroxypropyl methylcellulose.

9. A process according to claim 1, wherein the hydrophobic component is selected from the group consisting of ethyl cellulose, methacrylic acid polymers and copolymers, fatty acids and esters thereof, waxes and high molecular weight fatty alcohols.

10. A process according to claim 1, wherein the hydrophobic component is selected from the group consisting of EUDRAGIT NE 30 D from Rohm and Haas, stearic acid, behenic acid, glyceryl monostearate, glyceryl palmitostearate, glyceryl behenate, carbuna wax and cetyl alcohol.

11. A process according to claim 9, wherein the hydrophobic component is selected from the group consisting of cetyl alcohol and stearyl alcohol.

12. A process according to claim 1, wherein the poorly-compressible pharmaceutical agent comprises from about 10% to about 90% by weight of the formulation.

13. A process according to claim 1, wherein the hydrophilic erodible component comprises from about 10% to about 90% by weight of the formulation.

14. A process according to claim 1, wherein the hydrophobic component comprises from about 10% to about 30% by weight of the formulation.

15. A process according to claim 1, wherein the ratio of hydrophilic erodible component to hydrophobic component is 9:1 to 1:1.

16. A process according to claim 1, wherein the blend of step (a) comprises from about 40% to about 60% by weight of the poorly-compressible pharmaceutical agent and the hydrophilic erodible component and hydrophobic component are in a ratio of from 2:1 to 3:1.

17. A process according to claim 2, wherein the lubricant comprises about 0% to about 6% by weight of the blend.

18. A process for preparing an immediate-release pharmaceutical tablet formulation of a poorly-compressible pharmaceutical agent, which comprises the steps of:
(a) preparing a blend by combining the poorly-compressible pharmaceutical agent, a hydrophilic erodible component and a hydrophobic component, wherein the hydrophilic erodible component and the hydrophobic component are present in a ratio of 1:9 to 2:8; and
(b) compressing the blend into a tablet.

19. A process for preparing an sustained-release pharmaceutical tablet formulation of a poorly-compressible pharmaceutical agent, which comprises the steps of:
(a) preparing a blend by combining the poorly-compressible pharmaceutical agent, a hydrophilic erodible component and a hydrophobic component, wherein the hydrophilic erodible component and hydrophobic component are present in a ratio of 3:1 to 2:1; and
(b) compressing the blend into a tablet.

20. A process according to claim 1, wherein the tablet comprises about 500 mg of mefformin HCl.

21. A process according to claim 1, wherein the tablet comprises about 40-60 weight percent of mefformin HCl.

22. A pharmaceutical tablet prepared according to the process of claim 1.

23. A pharmaceutical tablet comprising 10% to about 90% by weight of a poorly-compressible pharmaceutical agent; from about 10% to about 90% by weight of hydrophilic erodible component; from about 10% to about 30% by weight of a hydrophobic component.

24. A pharmaceutical tablet comprising a poorly-compressible pharmaceutical agent, a hydrophilic erodible component and a hydrophobic component, wherein the ratio of hydrophilic erodible component to hydrophobic component is 9:1 to 1:1.

25. A pharmaceutical tablet comprising from about 40% to about 60% by weight of a poorly-compressible pharmaceutical agent, and a hydrophilic erodible component and a hydrophobic component, wherein the hydrophilic erodible component and hydrophobic component are present in a ratio of from 2:1 to 3:1.

26. An immediate-release pharmaceutical tablet formulation of a poorly-compressible pharmaceutical agent comprising a poorly-compressible pharmaceutical agent, a hydrophilic erodible component and a hydrophobic component, wherein the hydrophilic erodible component and hydrophobic component are present in a ratio of 1:9 to 2:8.

27. A sustained-release pharmaceutical tablet formulation of a poorly-compressible pharmaceutical agent comprising a poorly-compressible pharmaceutical agent, a hydrophilic erodible component and a hydrophobic component, wherein the hydrophilic erodible component and hydrophobic component are present in a ratio of 3:1 to 2:1.

28. A process for preparing a pharmaceutical tablet formulation of a pharmaceutical agent susceptible to hydrolysis and degradation due to water or a solvent, which comprises the steps of:
(a) preparing a blend by combining the poorly-compressible pharmaceutical agent, a hydrophilic erodible component and a hydrophobic component; and
(b) compressing the blend into a tablet. drugs susceptible to hydrolysis and degradation due to water or a solvent.

29. A pharmaceutical tablet prepared according to the process of claim 28.
